SEQUENCE LISTING (35 sequences)

SEQ ID NO: 1 - DNA, 26 bp, unidentified
  source 1..26: mol_type = genomic DNA
ccaatcggcactcatcctccagtgcc

SEQ ID NO: 2 - DNA, 25 bp, unidentified
  source 1..25: mol_type = genomic DNA
caatcggcactcatcctccagtgcc

SEQ ID NO: 3 - DNA, 24 bp, unidentified
  source 1..24: mol_type = genomic DNA
aatcggcactcatcctccagtgcc

SEQ ID NO: 4 - DNA, 29 bp, unidentified
  source 1..29: mol_type = genomic DNA
ccgtgacctcctactcacggctaaccctt

SEQ ID NO: 5 - DNA, 27 bp, unidentified
  source 1..27: mol_type = genomic DNA
gccaatcggcactcatcctccagtgcc

SEQ ID NO: 6 - DNA, 23 bp, unidentified
  source 1..23: mol_type = genomic DNA
gccaatcggcactcgatggctac

SEQ ID NO: 7 - DNA, 23 bp, unidentified
  source 1..23: mol_type = genomic DNA
gccaatcggcactcgatggctac

SEQ ID NO: 8 - DNA, 26 bp, unidentified
  source 1..26: mol_type = genomic DNA
ccaatcggcactcatcctccagtgcc

SEQ ID NO: 9 - DNA, 26 bp, unidentified
  source 1..26: mol_type = genomic DNA
caatccggcactcatcctccagtgcc

SEQ ID NO: 10 - DNA, 26 bp, unidentified
  source 1..26: mol_type = genomic DNA
aatcccggcactcatcctccagtgcc

SEQ ID NO: 11 - DNA, 25 bp, unidentified
  source 1..25: mol_type = genomic DNA
atcccaggcactcatcctccatgcc

SEQ ID NO: 12 - DNA, 26 bp, unidentified
  source 1..26: mol_type = genomic DNA
tcccaaggcactcatcctccagtgcc

SEQ ID NO: 13 - DNA, 26 bp, unidentified
  source 1..26: mol_type = genomic DNA
cccaatggcactcatcctccagtgcc

SEQ ID NO: 14 - DNA, 12 bp, unidentified
  source 1..12: mol_type = genomic DNA
ggttagggttag

SEQ ID NO: 15 - DNA, 26 bp, unidentified
  source 1..26: mol_type = genomic DNA
ccaatcggcactcatcctccagtgcc

SEQ ID NO: 16 - DNA, 18 bp, unidentified
  source 1..18: mol_type = genomic DNA
cggtgagatacgttggta

SEQ ID NO: 17 - DNA, 28 bp, unidentified
  source 1..28: mol_type = genomic DNA
agccaatcggcactcatcctccagtgcc

SEQ ID NO: 18 - DNA, 26 bp, unidentified
  source 1..26: mol_type = genomic DNA
ccgtgacctcctagtcacggctaacc

SEQ ID NO: 19 - DNA, 28 bp, unidentified
  source 1..28: mol_type = genomic DNA
agccaatcggcactcatcctccagtgcc

SEQ ID NO: 20 - DNA, 33 bp, unidentified
  source 1..33: mol_type = genomic DNA
accaatcggcactcatcctccagtgccgattgg

SEQ ID NO: 21 - DNA, 28 bp, unidentified
  source 1..28: mol_type = genomic DNA
agccaatcggcactcatcctccagtgcc

SEQ ID NO: 22 - DNA, 28 bp, unidentified
  source 1..28: mol_type = genomic DNA
agcaatccggcactcatcctccagtgcc

SEQ ID NO: 23 - DNA, 28 bp, unidentified
  source 1..28: mol_type = genomic DNA
agaatcccggcactcatcctccagtgcc

SEQ ID NO: 24 - DNA, 27 bp, unidentified
  source 1..27: mol_type = genomic DNA
agatcccaggcactcatcctccatgcc

SEQ ID NO: 25 - DNA, 28 bp, unidentified
  source 1..28: mol_type = genomic DNA
agtcccaaggcactcatcctccagtgcc

SEQ ID NO: 26 - DNA, 28 bp, unidentified
  source 1..28: mol_type = genomic DNA
agcccaatggcactcatcctccagtgcc

SEQ ID NO: 27 - DNA, 28 bp, unidentified
  source 1..28: mol_type = genomic DNA
agccaatcggcactcatcctccagtgcc

SEQ ID NO: 28 - DNA, 28 bp, unidentified
  source 1..28: mol_type = genomic DNA
agccaatcggcactcatcctccagtgcc

SEQ ID NO: 29 - DNA, 23 bp, unidentified
  source 1..23: mol_type = genomic DNA
ccaatcgccactctatgcaacca

SEQ ID NO: 30 - DNA, 23 bp, unidentified
  source 1..23: mol_type = genomic DNA
ccaatcgccactctatgcaacca

SEQ ID NO: 31 - DNA, 23 bp, unidentified
  source 1..23: mol_type = genomic DNA
caatccgccactctatgcaacca

SEQ ID NO: 32 - DNA, 23 bp, unidentified
  source 1..23: mol_type = genomic DNA
aatcccgccactctatgcaacca

SEQ ID NO: 33 - DNA, 23 bp, unidentified
  source 1..23: mol_type = genomic DNA
atcccagccactctatgcaacca

SEQ ID NO: 34 - DNA, 23 bp, unidentified
  source 1..23: mol_type = genomic DNA
tcccaagccactctatgcaacca

SEQ ID NO: 35 - DNA, 23 bp, unidentified
  source 1..23: mol_type = genomic DNA
cccaatgccactctatgcaacca

(19) United States
(12) Patent Application Publication
(10) Pub. No.: US 2026/0176681 A1
(43) Pub. Date: Jun. 25, 2026

(54) METHODS OF MEASURING ABSOLUTE LENGTH OF INDIVIDUAL TELOMERES

(71) Applicant: ARIZONA BOARD OF REGENTS ON BEHALF OF ARIZONA STATE UNIVERSITY, Scottsdale, AZ (US)

(72) Inventors: Julian CHEN, Chandler, AZ (US); Yang LI, Chandler, AZ (US); Dhenugen LOGESWARAN, Tempe, AZ (US)

(21) Appl. No.: 19/127,400

(22) PCT Filed: Nov. 3, 2023
(86) PCT No.: PCT/US2023/078733

§ 371 Date: May 5, 2025

Related U.S. Application Data
(60) Provisional application No. 63/382,486, filed on Nov. 4, 2022.

(51) Int. Cl. C12Q 1/6869; C12N 15/66; C12Q 1/6806; C12Q 1/6886

(52) U.S. Cl. CPC C12Q 1/6869; C12N 15/66; C12Q 1/6806; C12Q 1/6886

(57) ABSTRACT

The present disclosure relates to methods of directly measuring absolute length of individual telomeres without any PCR amplification or fluorescent probe hybridization. This method can be performed in a high-throughput 96-well plate format. The disclosure provides a method of processing genomic DNA samples comprising telomeric DNA to release the telomeric DNA from the non-telomeric genomic DNA that has one end tagged with a telomere adapter and the other end ligated with a DNA barcode adapter. The telomeric DNA samples are pooled and purified prior to long-read sequencing in certain embodiments, for example via a nanopore.

### CROSS-REFERENCE TO RELATED APPLICATIONS

This application claims the benefit of U.S. Provisional Application No. 63/382,486, entitled “Methods of Measuring Absolute Length Of Individual Telomeres,” which was filed Nov. 4, 2022, the entire disclosure of which is hereby incorporated herein by this reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 31,879-byte XML file named “11157-137_SeqList” created on Nov. 3, 2023.

### TECHNICAL FIELD

The present disclosure relates to methods of determining the absolute length of telomeres.

### BACKGROUND

Telomeres are specialized nucleoprotein structures that protect the ends of chromosomes in human cells from end-to-end fusions and are essential for maintaining genome integrity. The telomere DNA sequences consist of tracts of tandemly repeated sequences, (TTAGGG)n for all organisms in the kingdom Animalia and (TAGGG)n for insects. In human cells, the length of the telomere DNA varies from 5 to 15 Kb depending on the cellular age. The controlling mechanism of telomere length is not well known.

Telomere function and chromosome stability are essential for normal cell functioning and growth control. The telomere repeats are binding sites for specific proteins that distinguish the natural chromosome ends from DNA breaks. Telomeres shorten with each round of replication because of the “end-replication problem” resulting from the inability of conventional DNA polymerase to fully replicate the 3′-end of a linear DNA molecule. In normal cells, telomere shortening and/or dysfunction usually leads to chromosome fusions or cell senescence and eventually cell death. Telomere length therefore determines the replicative capacity in normal human cells and is viewed as a molecular clock that counts down as the cell ages. Remarkably, this cellular aging process can be stopped by ectopic expression of the telomerase enzyme.

Telomerase is the key enzyme responsible for infinite cell growth, as the maintenance of telomere length and integrity is essential for cell survival. Telomerase is a unique reverse transcriptase that specializes in telomeric DNA synthesis at chromosome ends. It contains two essential core components, the catalytic protein, telomerase reverse transcriptase (TERT) and the telomerase RNA (TR). The catalytic TERT protein synthesizes telomere DNA with a sequence, 5′-GGTTAG-3′, specified by a short template sequence in the intrinsic RNA component. This core ribonucleoprotein (RNP) complex associates with a number of accessory proteins, such as dyskerin, that play important roles in the biogenesis and regulation of the telomerase holoenzyme.

Telomerase activity is crucial for cellular immortality of stem cells and regulates the aging process. Deficiency or reduction of telomerase activity in stem cells results in premature aging phenotypes and bone marrow failure in patients with a “short telomere disorder” such as dyskeratosis congenita (DKC), aplastic anemia (AA), pulmonary fibrosis, Hoyeraal-Hreidarsson syndrome, acute myelogenous leukemia, and familial liver cirrhosis, those harbor mutations in telomerase genes. These mutations cause a reduction of telomerase activity, leading to a limited capacity for stem cell proliferation and low counts of blood cells or anemia. Families with these diseases show anticipation (the worsening of symptoms in subsequent generations) because of the progressive telomere shortening in each generation. Thus, having short telomeres is a risk factor of many genetic diseases caused by telomerase gene mutations.

In humans, telomerase is expressed in the germline and stem cells but not in normal somatic cells. Under normal circumstances, telomerase-negative cells only divide for a limited number of times, called the Hayflick limit, before they undergo senescence. The telomerase down-regulation and telomere shortening in normal human cells has also been proposed as a tumor suppressor mechanism that limits the growth potential of cancer cells. While repressed in most somatic cells, telomerase activity is abnormally up-regulated in most human tumors cells to maintain chromosome stability and infinite growth. About 85% of malignant tumors tested exhibit telomerase activity. In human tumor cells, the telomerase activity is up-regulated by expression of the hTERT mRNA and hTERT protein.

Thus, knowing the length of telomeres can have clinical diagnostic applications. It is clear that the ability to determine the length of telomeres accurately and in a timely manner will be an important tool for the early diagnosis of cancer and age-related diseases.

### SUMMARY OF THE DISCLOSURE

The disclosure relates to methods of directly measuring the absolute length of individual telomeres. The method is applicable for measuring the absolute length of individual telomeres in an individual cell as well as measuring the absolute length of individual telomeres from cells of a large population of individuals. Accordingly, the disclosure also provides a high-throughput method of identifying patients with short telomeres for early diagnosis and proper treatment.

The methods described herein comprise providing a genomic DNA sample, wherein the genomic DNA sample comprises telomeric DNA fragments, and ligating a telomere biotin-DNA adapter with the telomeric DNA fragments to produce a ligation product comprising biotinylated telomeric DNA fragments. The method further comprises performing a restriction enzyme digestion on the ligation product, wherein the biotinylated telomeric DNA fragments are released from non-telomeric genomic DNA in the ligation product. In certain aspects, the restriction enzyme is selected BamH-I, BcI-I, Bgl-II HF, and/or NotI. In some aspects, the biotinylated telomeric DNA fragments comprise sub-telomere sequences. The restriction enzyme used may be selected from 8-bp, 6-bp, or 4-bp cutters. In some aspects, the concentration of the telomere biotin-DNA adapter provided for the ligation reaction is 3-4 nM or about 3 nM in total.

The methods next comprise ligating a DNA barcode adapter to the biotinylated telomeric DNA fragments to produce barcoded and biotinylated telomeric DNA fragments. The DNA barcode prepares the biotinylated telomeric DNA fragments for DNA sequencing and to provides a method of identifying the specific genomic DNA sample in the sequencing results, as the method enables pooling the barcoded and biotinylated telomeric DNA fragments from a plurality of samples prior to sequencing. The DNA barcode adapter comprises a compatible sticky end to the end created from restriction enzyme digestion. Thus, the DNA barcode adapter is ligated to the sticky end created by restriction enzyme digestion (e.g., BamH-I, BcI-I, Bgl-II HF, or NotI).

The barcoded and biotinylated telomeric DNA fragments are purified using streptavidin beads that binds specifically to the biotin with an extremely high binding-affinity (KD=˜10⁻¹⁵ M) and then eluted from the streptavidin beads. In some implementations, the barcoded and biotinylated telomeric DNA fragments from multiple genomic DNA samples may be pooled prior to purification using streptavidin beads to produce eluted telomeric DNA fragments that would be finally processed for long-read sequencing.

The eluted telomeric DNA fragments are then ligated to a sequencing platform-specific adapter, for example, a nanopore-sequencing adapter or PacBio sequencer adapter, to prepare the eluted telomeric DNA fragments for sequencing. Next, the telomeric DNA fragments are sequenced using a long-read sequencing platform, wherein the read information generated is used to determine the absolute telomere length of each individual chromosome in the genomic DNA sample.

In a particular implementation, the method of determining absolute telomere length comprises providing a genomic DNA sample comprising telomeric DNA fragments and ligating a set of six telomere biotin-DNA adapters with the telomeric DNA fragments to produce a ligation product comprising biotinylated telomeric DNA fragments. The telomere biotin-DNA adapter comprises biotin attached via a cleavable spacer; a single-stranded 3′-overhang, which comprises a complementary to the 3′ end of the telomeric DNA fragments; and a 5′ phosphate for ligation with the telomeric DNA fragments. In some aspects, the telomeric biotin-DNA adapters are biotinylated DNA hairpins, wherein the biotinylated DNA hairpin has a stem-loop structure comprising a double-stranded stem and a single-stranded loop and the single-stranded 3′-overhang, wherein a biotin is attached at the 3′ end. In certain embodiments, the single-stranded 3′-overhang is at least 6 nucleotides in length. In other aspects, the telomeric biotin-DNA adapters are duplex DNAs, wherein the duplex DNA comprises a first oligonucleotide and a second oligonucleotide, and the duplex DNA comprises an annealed region of 12-18 bp, wherein the first oligonucleotides and the second oligonucleotide are perfectly complementary.

The Tₘ of the annealed region is greater than 30° C. In some aspects, the second oligonucleotide is longer than the first oligonucleotide thereby providing the single-stranded 3′-overhang. In some aspects, the first oligonucleotide comprises a 5′ phosphate. In certain embodiments of the duplex DNA adapter, the biotin is attached to the 3′ end of the first oligonucleotide. In other embodiments of the duplex DNA adapter, the biotin is attached to the 5′ end of the second oligonucleotide.

The set of six telomere biotin-DNA adapters comprise: a first telomere adapter with its single-stranded 3′-overhang comprising CCAATC (3′ to 5′); a second telomere adapter with its single-stranded 3′-overhang comprising CAATCC (3′ to 5′); a third telomere adapter with its single-stranded 3′-overhang comprising AATCCC (3′ to 5′); a fourth telomere adapter with its single-stranded 3′-overhang comprising ATCCCA (3′ to 5′); a fifth telomere adapter its single-stranded 3′-overhang comprising TCCCAA (3′ to 5′); and a sixth telomere adapter with its single-stranded 3′-overhang comprising CCCAAT (3′ to 5′).

In some embodiments, the first telomere adapter comprises the sequence set forth in SEQ ID NO. 8 or has the sequence set forth in SEQ ID NO. 21; the second telomere adapter comprises the sequence set forth in SEQ ID NO. 9 or has the sequence set forth in SEQ ID NO. 22; the third telomere adapter comprises the sequence set forth in SEQ ID NO. 10 or has the sequence set forth in SEQ ID NO. 23; the fourth telomere adapter comprises the sequence set forth in SEQ ID NO. 11 or has the sequence set forth in SEQ ID NO. 24; the fifth telomere adapter comprises the sequence set forth in SEQ ID NO. 12 or has the sequence set forth in SEQ ID NO. 25; and the sixth telomere adapter comprises the sequence set forth in SEQ ID NO. 13 or has the sequence set forth in SEQ ID NO. 26.

The method further comprises digesting the ligation product with a restriction enzyme to release the biotinylated telomeric DNA fragments from the non-telomeric genomic DNA; ligating a DNA barcode adapter to the biotinylated telomeric DNA fragments at the restriction enzyme digestion site to produce barcoded and biotinylated telomeric DNA fragments; cleaving the biotin from the barcoded and biotinylated telomeric DNA fragments to produce barcoded telomeric DNA fragments; and sequencing the barcoded telomeric DNA fragments. In some aspects, the barcoded telomeric DNA fragments are sequenced using nanopore sequencing. In such implementations, the method further comprises determining the absolute length of telomeres in the genomic DNA sample from the nanopore sequencing results.

In another implementation, the method of determining the absolute telomere length in a single cell comprises isolating genomic DNA from a single cell to provide a genomic DNA sample comprising telomeric DNA fragments and ligating a set of six telomere adapters with the telomeric DNA fragments to produce a ligation product comprising biotinylated telomeric DNA fragments, wherein the telomere adapter comprises a single-stranded 3′-overhang, which comprises a complementary to the 3′ end of the telomeric DNA fragments and a 5′ phosphate for ligation with the telomeric DNA fragments. The set of six telomere adapters comprise a first telomere adapter with its single-stranded 3′-overhang comprising CCAATC (3′ to 5′); a second telomere adapter with its single-stranded 3′-overhang comprising CAATCC (3′ to 5′); a third telomere adapter with its single-stranded 3′-overhang comprising AATCCC (3′ to 5′); a fourth telomere adapter with its single-stranded 3′-overhang comprising ATCCCA (3′ to 5′); a fifth telomere adapter its single-stranded 3′-overhang comprising TCCCAA (3′ to 5′); and a sixth telomere adapter with its single-stranded 3′-overhang comprising CCCAAT (3′ to 5′).

The method further comprises digesting the ligation product with a restriction enzyme to release the biotinylated telomeric DNA fragments from the non-telomeric genomic DNA; ligating a DNA barcode adapter to the biotinylated telomeric DNA fragments at the restriction enzyme digestion site to produce barcoded and biotinylated telomeric DNA fragments; amplifying the barcoded and biotinylated telomeric DNA fragments with phi29 DNA polymerase with a forward primer comprising a sequence complementary to the DNA barcode adapter, and a reverse primer comprising a sequence complementary a portion of the telomere adapter lacking single-stranded 3′-overhang; and sequencing the amplified telomeric DNA fragments. In some aspects, the amplified telomeric DNA fragments are sequenced using nanopore sequencing. In such implementations, the method further comprises determining the absolute length of telomeres in the genomic DNA sample from the nanopore sequencing results.

Where the steps of ligating the biotinylated DNA hairpins, digesting the ligation product, and ligating the DNA barcode are performed in a single reaction vessel. Where the telomeric adapters are DNA hairpins, the DNA hairpin has a stem-loop structure comprising a double-stranded stem and a single-stranded loop and the single-stranded 3′-overhang. In some aspects, the single-stranded 3′-overhang is at least 6 nucleotides in length. In a particular embodiment, the first telomere adapter comprises a sequence set forth in SEQ ID NO. 8 or has a sequence set forth in SEQ ID NO. 21; the second telomere adapter comprises a sequence set forth in SEQ ID NO. 9 or has a sequence set forth in SEQ ID NO. 22; the third telomere adapter comprises a sequence set forth in SEQ ID NO. 10 or has a sequence set forth in SEQ ID NO. 23; the fourth telomere adapter comprises a sequence set forth in SEQ ID NO. 11 or has a sequence set forth in SEQ ID NO. 24; the fifth telomere adapter comprises a sequence set forth in SEQ ID NO. 12 or has a sequence set forth in SEQ ID NO. 25; and the sixth telomere adapter comprises a sequence set forth in SEQ ID NO. 13 or has a sequence set forth in SEQ ID NO. 26.

In certain aspects, the telomeric adapters are duplex DNAs, wherein the duplex DNA comprises a first oligonucleotide and a second oligonucleotide, wherein the duplex DNA comprises an annealed region of 12-18 bp, wherein the first oligonucleotides and the second oligonucleotide are perfectly complementary, and the second oligonucleotide is longer than the first oligonucleotide thereby providing the single-stranded 3′-overhang. The Tₘ of the annealed region is >30° C. In some aspects, the first oligonucleotide comprises a 5′ phosphate. In certain implementations, the biotin is attached to the 3′ end of the first oligonucleotide. In other implementations, the biotin is attached to the 5′ end of the second oligonucleotide.

For the method of determining absolute telomere length where a set of six telomere biotin-DNA adapters or a set of six telomeric adapters are used for ligation with telomeric DNA fragments, the concentrations of the concentrations of the individual telomere biotin-DNA adapter or the individual telomere adapters are the same, for example, between 0.25-0.70 nM. In some implementations, the total concentration of six telomere biotin-DNA adapters or the total concentration of the six telomere adapters is 3-4 nM.

### BRIEF DESCRIPTION OF THE DRAWINGS

FIG. 1 depicts, in accordance with certain embodiments, a schematic of the method of measuring absolute length of individual telomeres using telomere adapters with a hairpin structure and long read sequencing via a nanopore. The sequence of exemplary hairpin adapter (ASU-7530) is set forth in SEQ ID NO. 1.

FIG. 2 depicts, in accordance with certain embodiments, certain optimal 3′-end overhang length in the hairpin adapter for telomere DNA ligation and the optimal temperature for hairpin adapter ligation. The sequence of ASU-7530 is set forth in SEQ ID NO. 1. The sequence of ASU-7540 is set forth in SEQ ID NO. 2. The sequence of ASU-7541 is set forth in SEQ ID NO. 3. ASU-7530, ASU-7540 and ASU-7541 respectively have 6 nt, 5 nt and 4 nt 3′ overhangs for base pairing with a target ssDNA molecule.

FIG. 3 depicts, in accordance with certain embodiments, the minimal concentration of hairpin adaptor (ASU-7420) required for telomere DNA ligation.

FIG. 4 depicts, in accordance with certain embodiments, the design of telomere biotin-DNA adapters, which may be a hairpin or a duplex. Embodiments of designs 1 and 2 of the telomere biotin-DNA adapter are biotinylated at 3′-end and phosphorylated at 5′-end. The telomere-specific adapters comprise a 6-nucleotide sequence complementary to a tract of telomere repeats, which would be ligated to the telomeric fragments in a genomic DNA sample and contain a photocleavable spacer which allows gentle elution of the ligated telomeric DNA fragments and removal of the bulky biotin.

FIG. 5 depicts, in accordance with certain embodiments, the set of six telomere biotin-DNA adapters for ligation to telomeric fragments in a genomic DNA sample.

FIG. 6 depicts, in accordance with certain embodiments, the nearly 100% efficient ligation and streptavidin beads enrichment of a telomere like radiolabeled oligonucleotide. Further, the optimal ultraviolet (UV) wavelength and UV exposure time to achieve efficient photocleavage following streptavidin beads enrichment. All conditions tested using an exemplary hairpin adapter (ASU-8389) required for telomere ligation. Schematics of the substrate (radiolabeled ASU-4884) and hairpin (ASU-8389) ligated DNA products are shown in the right panel indicating their respective positions in the gel.

FIG. 7 depicts, in accordance with certain embodiments, the comparison of a duplex DNA adapter and a hairpin adapter for ligation to a telomere like oligonucleotide followed by streptavidin bead enrichment and photocleavage.

FIG. 8A depicts, in accordance with certain embodiments enrichment of human telomeres following hairpin adapter ligation. FIG. 8B depicts, in accordance with certain embodiments, a schematic of the set of six duplex adapter that was tested.

FIG. 9 depicts an exemplary graph illustrating the change in telomeric length as people age with lines indicating the telomere length at the 99th percentile, 90th percentile, 50th percentile, 10th percentile, and 1st percentile.

FIG. 10 depicts, in accordance with certain embodiments, a method of increasing the amount of telomeric DNA fragments isolated from a single cell to enable long-read sequencing using amplification with phi29 DNA polymerase. The forward primer for the amplification reaction has a sequence complementary to the DNA barcode adapter, while the reverse primer has a sequence complementary the telomere adapter (minus the sequence of the single-stranded 3′-overhang).

FIG. 11 shows a bar plot of estimated bases collected during a nanopore telomere sequencing run (Y axis) vs estimated read length in kilobases (X axis). The tip of each bar is in red which shows that the nanopore is blocked by a read. A representative read in the lower panel shows premature truncation of the read with the region of the hairpin duplex highlighted. The read terminates within the loop of this hairpin. Thus, it is very likely due to the lower unwinding efficiency of the hairpin duplex or the presence of the biotin group at the 3′ end physically restricting the rest of the sequence to travel through the nanopore and be sequenced. The introduction of a photocleavable group to cleave away the biotin and use a duplex adapter as opposed to a hairpin adapter will solve this issue.

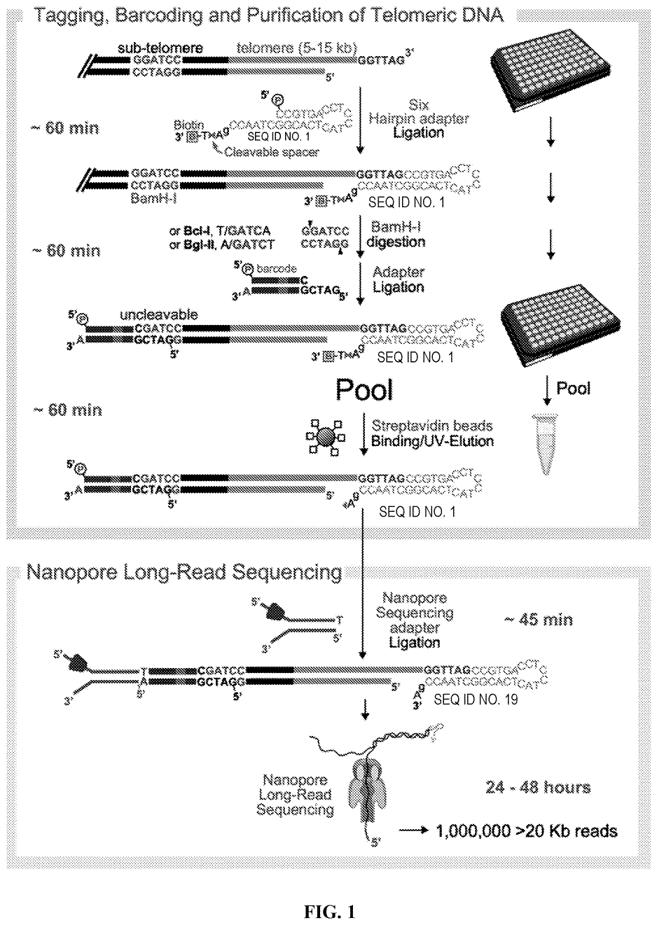

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

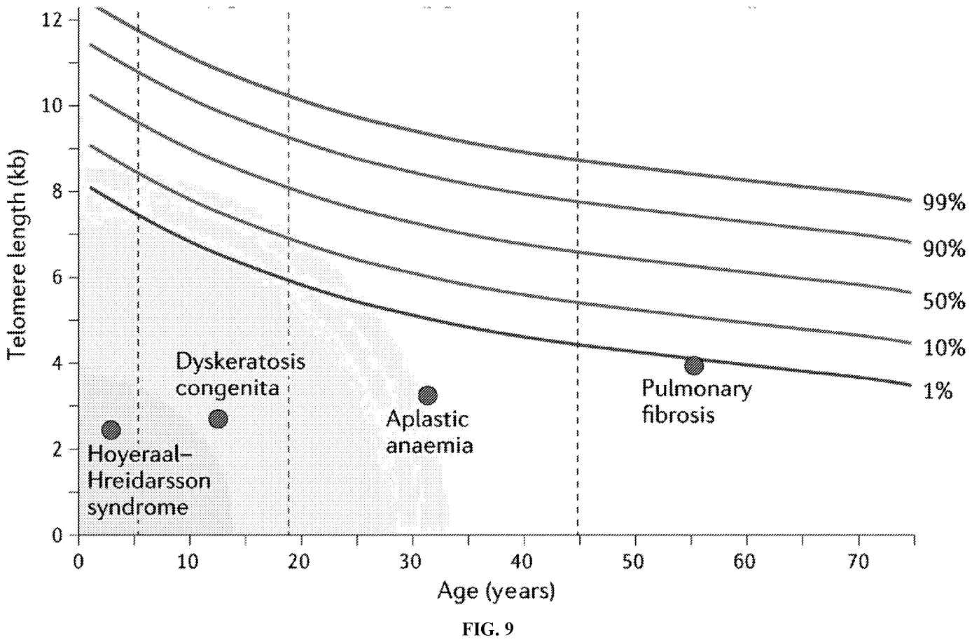

This disclosure is directed to a method of directly measuring absolute length of individual telomeres. In some aspects, the method enables rapid measurement of absolute length of individual telomeres from a large population to identify patients with short telomeres. Accordingly, also described herein are methods of identifying subjects with short telomeres. As used herein, the term “short telomeres” refers telomeres lengths of less than 8 kb, less than 5 kb, or less than 4 kb. In some aspects, the term “short telomeres” refers to telomeres of length than is below 1 percentile for a subject's age group (see FIG. 9). In other aspects, the method enables rapid measurement of absolute length of individual telomeres from a single cell.

The described method uses biotinylated adapters to tag telomeric DNA for affinity-purification, which are then barcoded to facilitating sequencing and subsequent sequencing data analysis. The method described herein measures absolute length of individual telomeres by directly sequencing each telomeric DNA fragment in the sample, which is unlike any method of measuring telomere length in the prior art. Many of the currently available telomere length measurement techniques require PCR amplification using telomeric primers that target anywhere in the telomeric sequence, which often leads to inaccuracy of the ensemble average telomere length in a DNA sample (Table 1). The method described herein is also fully scalable to be cost-effective compared to many fluorescent probe hybridization methods such as the popular flow-FISH and Q-FISH assays.

TABLE 1
Comparison of commonly used methods for
measuring relative telomere length (TL)

| Method | Detection | Pros | Cons |
|---|---|---|---|
| TRF | Radioactivity | Gold standard | Cannot measure shortest |
| | | | TL |
| | | | Labor intensive |
| qPCR | Fluorescence | Low input | Cannot measure absolute |
| | | | TL |
| | | High throughput | Error prone |
| Q-FISH | Fluorescence | Highly sensitive | Very labor-intensive |
| | | Measures individual | Does not detect very |
| | | TL | short telomeres |
| FLOW- | Fluorescence | Specific cell types | Expensive |
| FISH | | sorted by flow | Limited to PBMCs |
| | | cytometry | |
| Luminex | Fluorescence | No amplification | provides only relative TL |

TRF: Telomere restriction fragment assay;
Q-FISH: Quantitative fluorescence in-situ hybridization

The method of determining absolute length of individual telomeres comprises providing a genomic DNA sample, wherein the genomic DNA sample comprises telomeric DNA fragments, and ligating a telomere biotin-DNA adapter with the telomeric DNA fragments to produce a ligation product comprising biotinylated telomeric DNA fragments. The concentration of the telomere biotin-DNA adapter is the ligation reaction is 3-4 nM. The method further comprises performing a restriction enzyme digestion on the ligation product, wherein the biotinylated telomeric DNA fragments are released from non-telomeric genomic DNA in the ligation product. In some aspects, the biotinylated telomeric DNA fragments comprise sub-telomere sequences. In some implementations, the biotinylated telomeric DNA fragments comprise between 20-50 kb of sub-telomere sequences. The sequence information from the sub-telomere sequences can increase the accuracy in which the absolute length of individual telomeres are attributed to either the p arm or the q arm of a specific chromosome. The restriction enzyme used may be selected from 8-bp, 6-bp, or 4-bp cutters. For generating biotinylated telomeric DNA fragments comprises more sub-telomere sequences, an 8-bp cutter or a 6-bp cutter is preferred for the restriction enzyme digestion. In certain implementations, the restriction enzyme used is selected from the group consisting of: BamH-I, BcI-I, Bgl-II HF, and NotI.

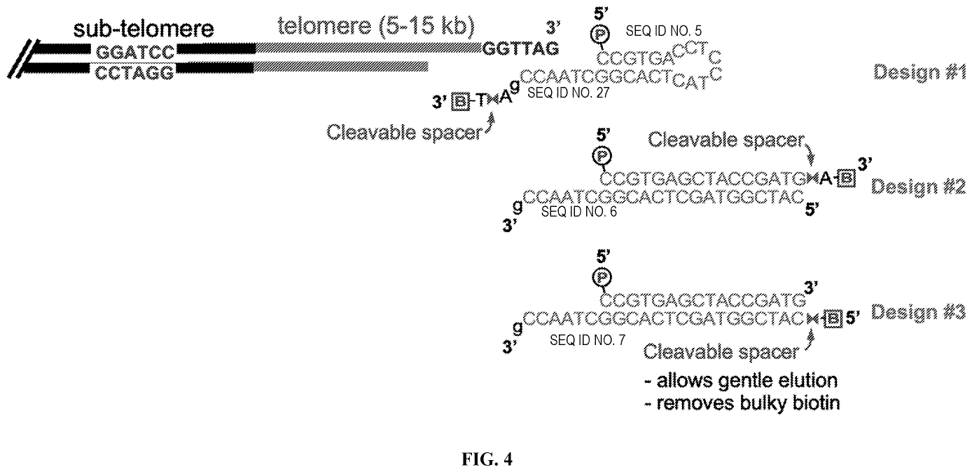

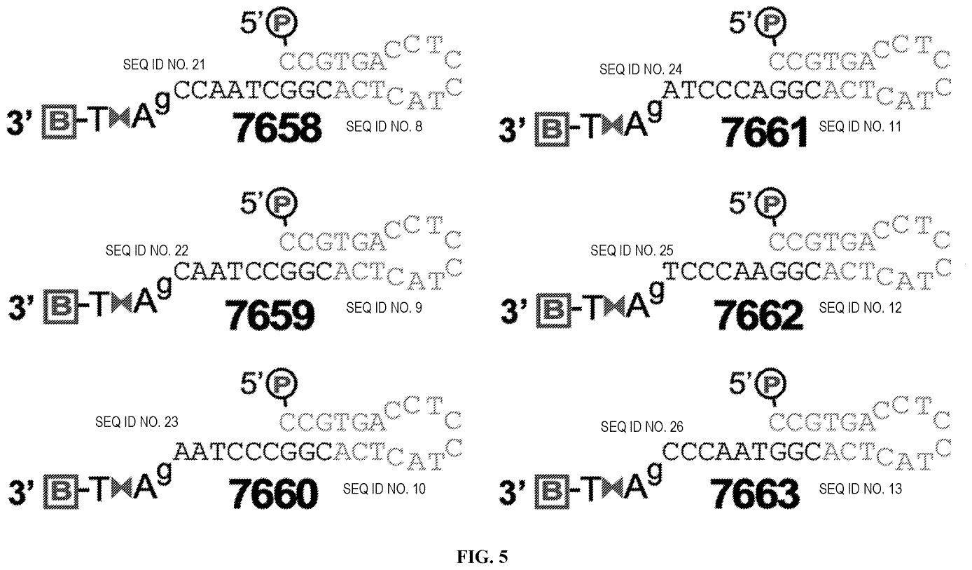

The telomere biotin-DNA adapters are biotinylated and comprise a single-stranded 3′-overhang, which comprises a 6-nt sequence complementary to the 3′ end of the telomeric DNA fragments (FIG. 4). The single-stranded 3′ overhang is ligated to the telomeric DNA fragments in the genomic DNA sample. Thus, in some aspects, the 6-nt sequence complementary to the 3′ end of the telomeric DNA fragments forms the single-stranded 3′-overhang or a part of the single-stranded 3′-overhang of the telomere adapter. In preferred embodiments, the biotin is attached to either the 3′ end or 5′ end of the telomere biotin-DNA adapter via a cleavable spacer. In some aspects, a cleavable spacer is present in the loop region of the telomere biotin-DNA adapter. In some embodiments, the cleavable spacer in the loop region of the adapter is a second spacer in the telomere biotin-DNA adapter. In other embodiments, the cleavable spacer in the loop region of the adapter is the sole spacer in the telomere biotin-DNA adapter. In particular embodiments, the cleavable spacer is photocleavable, for example, cleavable by 300 nm UV light. It is important that the photocleavable spacer is not cleaved by a wavelength of light such as 254 nm or shorter that causes DNA damage.

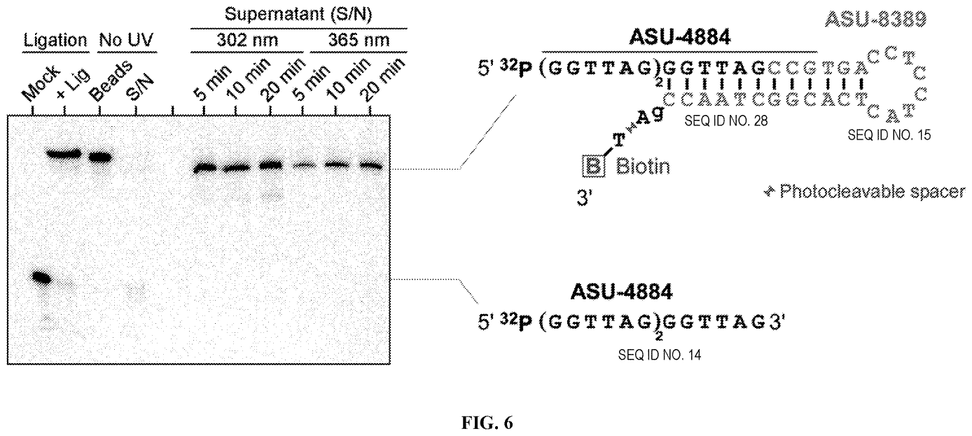

To ensure all telomeric DNA fragments in the genomic DNA sample from an organism are biotinylated, a set of telomere biotin-DNA adapters should be ligated with the telomeric DNA fragments wherein the 6-nt sequence complementary to the 3′ end of the telomeric DNA fragments of the set of telomere adapters encompass all possible permutations of the terminal sequences of telomeres of the organism. Thus, to ensure all telomeric DNA fragments in the genomic DNA sample from organism in the kingdom Animalia are biotinylated, a set of six telomere adapters is used for the ligation reaction. For the ligation reaction, the concentration of each of the telomere adapters is the same, for example, between 0.25 nM and 0.67 nM. In some aspects, the total concentration of the six telomere adapters is 3-4 nM in the ligation reaction. The 6-nt sequence complementary to the 3′ end of the telomeric DNA fragments of set of telomere biotin-DNA adapters encompass all six possible permutations of terminal sequences of telomeres: GGTTAG, GTTAGG, TTAGGG, TAGGGT, AGGGTT and GGGTTA. Accordingly, in some implementations, the method comprises ligating the telomeric DNA fragments with a set of six telomere biotin-DNA adapters to produce a ligation product comprising biotinylated telomeric DNA fragments from all telomere sequences found in the genomic DNA sample. The set of six telomere adapters comprise a first telomere adapter with 6-nt sequence complementary to the 3′ end of the telomeric DNA fragments comprising CCAATC (3′ to 5′), a second telomere adapter with 6-nt sequence complementary to the 3′ end of the telomeric DNA fragments comprising CAATCC (3′ to 5′), a third telomere adapter with 6-nt sequence complementary to the 3′ end of the telomeric DNA fragments comprising AATCCC (3′ to 5′), a fourth telomere adapter with 6-nt sequence complementary to the 3′ end of the telomeric DNA fragments comprising ATCCCA (3′ to 5′), a fifth telomere adapter with 6-nt sequence complementary to the 3′ end of the telomeric DNA fragments comprising TCCCAA (3′ to 5′), and a sixth telomere adapter with 6-nt sequence complementary to the 3′ end of the telomeric DNA fragments comprising CCCAAT (3′ to 5′). In some implementations the first telomere adapter comprises a sequence set forth in SEQ ID NO. 8; the second telomere adapter comprises a sequence set forth in SEQ ID NO. 9; the third telomere adapter comprises a sequence set forth in SEQ ID NO. 10; the fourth telomere adapter comprises a sequence set forth in SEQ ID NO. 11; the fifth telomere adapter comprises a sequence set forth in SEQ ID NO. 12; and the sixth telomere adapter comprises a sequence set forth in SEQ ID NO. 13. In certain implementations, the set of six telomere biotin-DNA adapters is the group of telomere biotin-DNA adapters shown in FIG. 5.

In some aspects, the telomere adapter is a biotinylated DNA hairpin. The biotinylated DNA hairpin has a stem-loop structure comprising a double-stranded stem and a single-stranded loop and a single-stranded 3′-overhang, wherein the biotin is attached at the 3′ end. The biotinylated DNA hairpin comprises a sequence that is complementary to the 3′ end of the telomeric DNA fragments, which is located within the single-stranded 3′-overhang, and a 5′ phosphate to allow for ligation with the telomeric DNA fragments. Thus, the single-stranded 3′-overhang is at least 6 nucleotides in length. FIG. 5 depicts an exemplary set of six telomere biotin-DNA adapters for producing biotinylated DNA biotinylated telomeric DNA fragments from all telomeric fragments in the genomic DNA sample. Where the cleavable spacer in the loop region of the telomere adapter is the sole spacer in the telomere adapter, the biotin is attached to the telomere adapter via the cleavable spacer in the loop region of the telomere adapter. Where the cleavable spacer in the loop region of the telomere adapter is the second spacer in the telomere adapter, the biotin is attached at an end of the telomere adapter via the cleavable spacer.

In some aspects, the telomere biotin-DNA adapter is a duplex DNA adapter (see, for example, designs #2 and #3 in FIG. 4), wherein the telomere biotin-DNA adapter comprises a first oligonucleotide and a second oligonucleotide, where the first oligonucleotides and the second oligonucleotide are perfectly complementary over 12-18 contiguous nucleotides and the second oligonucleotide is longer than the first oligonucleotide. Thus, the duplex DNA adapter comprises an annealed region of 12-18 bp and a single-stranded 3′-overhang. In some aspects, the single-stranded 3′-overhang is at least 6 nucleotides long. The Tₘ of the annealed region is >30° C. In particular embodiments, the annealed region of the duplex DNA 17 bp in length.

The first oligonucleotide of the duplex DNA adapter forms the sense strand (5′ to 3′ from left to right) and the second oligonucleotide of the duplex DNA form the antisense strand (3′ to 5′ from left to right) with the second oligonucleotide further comprising the 6-nt sequence complementary to the 3′ end of the telomeric DNA fragments, which forms the single-stranded 3′-overhang of the telomere adapter. As such, first oligonucleotide comprises a 5′ phosphate to allow the duplex DNA adapter to ligate with the telomeric DNA fragments. In some implementations, the biotin is attached to the 3′ end of the first oligonucleotide. In other implementations, the biotin is attached to the 5′ end of the second oligonucleotide. The most cost-effective telomere adapter design would be a duplex DNA with the biotin attached to the 3′ end of the first oligonucleotide.

The method next comprises ligating a DNA barcode adapter to the biotinylated telomeric DNA fragments to produce barcoded and biotinylated telomeric DNA fragments. The DNA barcode prepares the biotinylated telomeric DNA fragments for DNA sequencing and to provides a method of identifying the specific genomic DNA sample in the sequencing results, as the method enables pooling the barcoded and biotinylated telomeric DNA fragments from a plurality of samples prior to sequencing. The DNA barcode adapter comprises a compatible sticky end to the end created from restriction enzyme digestion. Thus, the DNA barcode adapter is ligated to the sticky end created by restriction enzyme digestion. In some aspects, the DNA barcode adapter is at least 6 bp in length. In some implementations, the DNA barcode adapter comprises a barcoding sequence along with the compatible sticky end to the end created from restriction enzyme digestion.

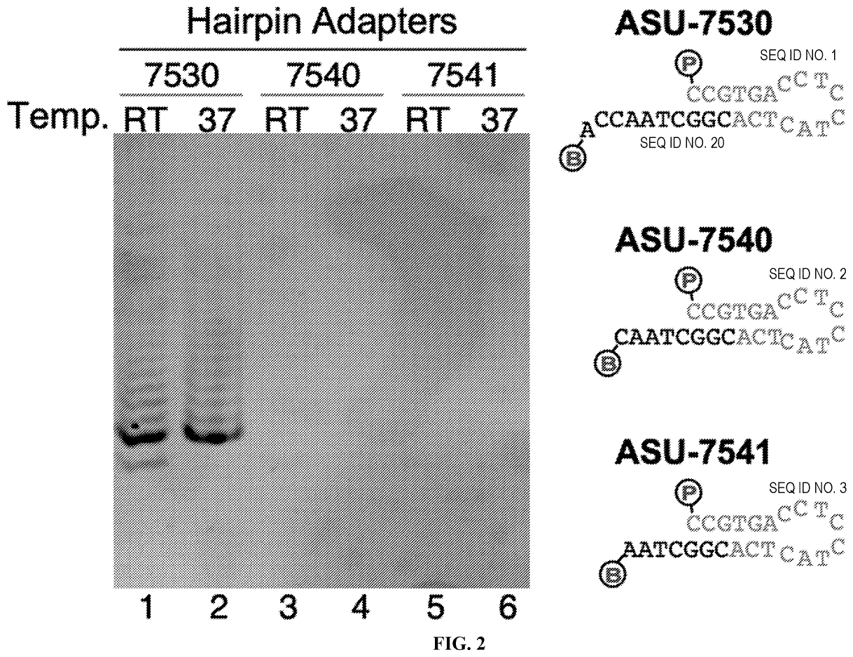

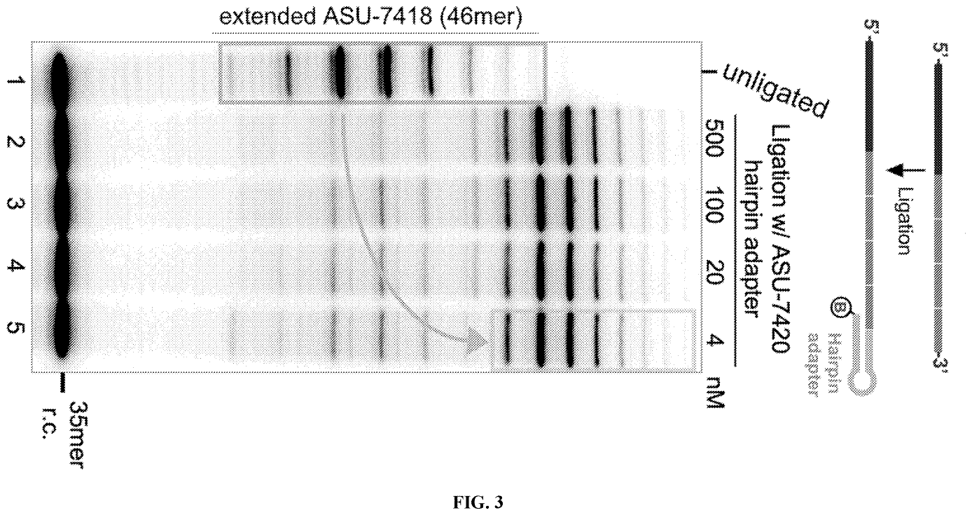

As shown in FIG. 2, at least 6 bp hybrid between the biotinylated DNA hairpin telomere adapter and the substrate is required for successful ligation. After telomere adapter ligation to a ssDNA substrate followed by PCR amplification of the ligated DNA shows that only ASU-7530 is capable of being efficiently ligated and detected by gel electrophoresis (lanes 1 and 2) but not ASU-7540 and ASU-7541 (lanes 3-6). FIG. 2 also shows that ligation is equally successful at both room temperature and 37° C. Thus, ligation can be performed at elevated temperatures for increased specificity and ligation efficiency. As shown in FIG. 3, the lowest concentration of biotinylated DNA hairpin telomere adapter tested in the experiment (at 4 nM) shows an upward shift of substrate DNA following ligation (lane 5). This is likely due to increased molecular weight. Increasing the biotinylated DNA hairpin telomere adapter concentration above 4 nM does not improve the shift significantly and thus concluding 4 nM hairpin adapter is sufficient to achieve complete ligation. Thus in some aspects, the concentration of the hairpin adapters for use in the ligation step is 3-4 nM. Where a set of six adapters are used for the telomere adapter ligation, the concentration of the individual adapters are the same and each at 0.50-0.67 nM. In some implementations, the concentration of the individual adapters are each at 0.25-0.50 nM or about 0.25 nM.

The barcoded and biotinylated telomeric DNA fragments are purified using streptavidin beads that binds specifically to the biotin with an extremely high binding-affinity (KD=˜10⁻¹⁵ M) and then eluted from the streptavidin beads. In some implementations, where the telomere biotin-DNA adapter comprises a biotin attached via cleavable spacer, the barcoded and biotinylated telomeric DNA fragments are purified using streptavidin beads that binds specifically to the biotin with an extremely high binding-affinity and then eluted from the streptavidin beads through 300 nm UV-irradiation to cleave the cleavable spacer so that eluted telomeric DNA fragments prepared for long-read sequencing are not biotinylated. As the presence of biotin can impair passage of DNA strands through the nanopore, it is preferrable for the biotin to be removed from the telomeric DNA fragments prior to nanopore sequencing.

In some implementations, the barcoded and biotinylated telomeric DNA fragments from multiple genomic DNA samples may be pooled prior to purification using streptavidin beads to produce eluted telomeric DNA fragments that would be finally processed for long-read sequencing. Depending on the volume of the pooled barcoded and biotinylated telomeric DNA fragments, it may be necessary to concentrate the pooled barcoded and biotinylated telomeric DNA fragments (reduce the pooled volume) and remove the free biotin DNA adapter prior to binding with streptavidin beads. Where the total number of samples is small, for example less than 12 samples, purification using with AMPure XP beads would be sufficient to remove the free biotin DNA adapter. Where the total number of samples is greater than 12, for example between 12-96, then it would be preferrable to reduce the total volume of the pooled barcoded and biotinylated telomeric DNA fragments by ethanol precipitation prior to purification using AMPure XP beads.

The eluted telomeric DNA fragments are then ligated to a sequencing platform-specific adapter, for example, a nanopore-sequencing adapter or PacBio sequencer adapter, to prepare the eluted telomeric DNA fragments for sequencing. Next, the telomeric DNA fragments are sequenced using a long-read sequencing platform, wherein the read information generated is used to determine the absolute telomere length of each individual chromosome in the genomic DNA sample. Each nanopore sequencing analysis costs around $500 and could measure telomere absolute length in the DNA samples pooled from >100 individuals, which makes the described method extremely cost-effective and competitive compared to the existing technologies for measuring telomere length on the market. In certain implementations, the DNA library will be loaded to the nanopore flow cell (R9.4.1) that contains up to 512 nanopore channels. Sequencing reads will then be collected over 48-72 hours with a yield of over 1 million reads of >20 kb long DNA sequence. Different types of flow cells can be used for different throughputs.

In some implementations, the read information from nanopore sequencing is analyzed using a computational program to decode the barcoded reads and to determine the telomere absolute length of each individual chromosome in each DNA sample. In some aspects, the barcoded reads will be sorted and assigned to each of the samples, and each read will be assigned to either p or q arms of each specific chromosome in each DNA sample.

The genomic DNA sample provided to determining absolute telomere length may be isolated from any tissues including whole blood, peripheral blood mononuclear cells, buccal swaps, or any method or form of genomic DNA sample collection or from any eukaryotic species including humans.

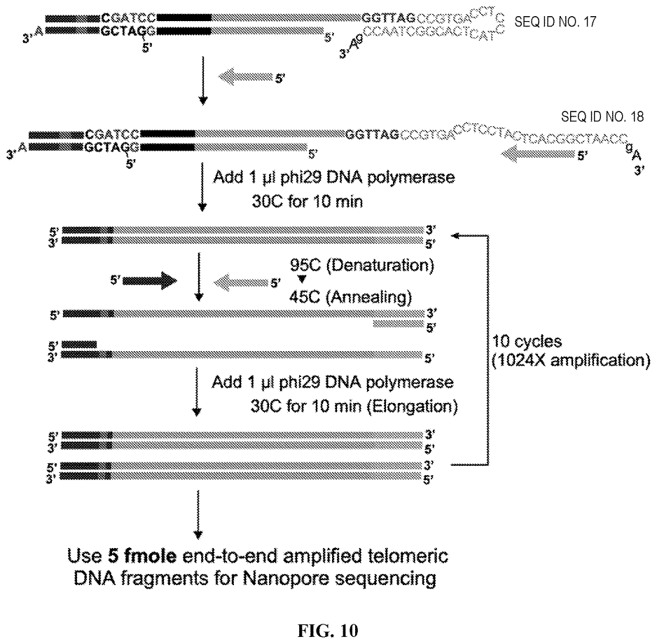

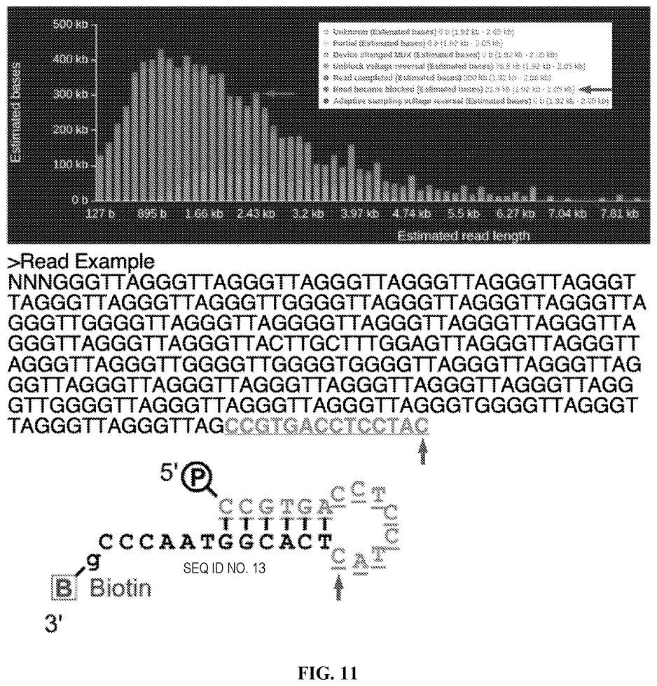

For the method of determining absolute telomere length in a single cell, the method comprises isolating genomic DNA from a single cell, wherein the isolated genomic DNA comprises telomeric DNA fragments, and ligating the telomeric DNA fragments with a set of telomere adapters to produce a ligation product comprising biotinylated telomeric DNA fragments. In some embodiments, the telomere adapters may be the telomere biotin-DNA adapter described above and in FIG. 4. In other embodiments, the telomere adapter differs structurally from the designs shown in FIG. 4 by the lack of a biotin attachment (see exemplary telomere adapter shown in FIG. 10). For a single cell from an organism in the kingdom Animalia, a set of six telomere adapters are ligated with the telomeric DNA fragments. The set of six telomere adapters comprise a first telomere adapter with 6-nt sequence complementary to the 3′ end of the telomeric DNA fragments comprising CCAATC (3′ to 5′), a second telomere adapter with 6-nt sequence complementary to the 3′ end of the telomeric DNA fragments comprising CAATCC (3′ to 5′), a third telomere adapter with 6-nt sequence complementary to the 3′ end of the telomeric DNA fragments comprising AATCCC (3′ to 5′), a fourth telomere adapter with 6-nt sequence complementary to the 3′ end of the telomeric DNA fragments comprising ATCCCA (3′ to 5′), a fifth telomere adapter with 6-nt sequence complementary to the 3′ end of the telomeric DNA fragments comprising TCCCAA (3′ to 5′), and a sixth telomere adapter with 6-nt sequence complementary to the 3′ end of the telomeric DNA fragments comprising CCCAAT (3′ to 5′). Thus, all telomeric DNA fragments within the genomic DNA sample of the organism in the kingdom Animalia are biotinylated.

The method next comprises performing a restriction enzyme digestion on the ligation product, wherein the biotinylated telomeric DNA fragments are released from non-telomeric genomic DNA in the ligation product. The restriction enzyme used may be selected from 8-bp, 6-bp, or 4-bp cutters. For generating biotinylated telomeric DNA fragments comprises more sub-telomere sequences, an 8-bp cutter or a 6-bp cutter is preferred for the restriction enzyme digestion. In certain implementations, the restriction enzyme used is selected from the group consisting of: BamH-I, BcI-I, Bgl-II HF, and NotI. The method further comprises ligating a DNA barcode to the biotinylated telomeric DNA fragments to produce barcoded and biotinylated telomeric DNA fragments. In some implementations, the ligation steps and the restriction enzyme digestion step are performed in a one-pot reaction.

In some implementations, barcoded and biotinylated telomeric DNA fragments from a plurality of single cell samples are pooled together and then purified so that a sufficient amount of telomeric DNA fragments are collected for long-read sequencing. For nanopore long-read sequencing, at least 5 fmole of telomeric DNA fragments need to be provided for sequencing. In some aspects, the pooled barcoded and biotinylated telomeric DNA fragments are purified using streptavidin beads that binds specifically to the biotin with an extremely high binding-affinity and then eluted from the streptavidin beads. Depending on the volume of the pooled barcoded and biotinylated telomeric DNA fragments, it may be necessary to concentrate the pooled barcoded and biotinylated telomeric DNA fragments (reduce the pooled volume) prior to elution with streptavidin beads. Ethanol precipitation or purification with AMPure XP beads may be reduced to reduce the total volume of the pooled barcoded and biotinylated telomeric DNA fragments.

Where the telomere adapter comprises a biotin attached via cleavable spacer, the barcoded and biotinylated telomeric DNA fragments are purified using streptavidin beads that binds specifically to the biotin with an extremely high binding-affinity and then eluted from the streptavidin beads through UV irradiation at the appropriate wavelength to cleave the cleavable spacer so that the eluted telomeric DNA fragments prepared for long-read sequencing are not biotinylated. In particular implementations, 300 nm UV-irradiation is used to cleave the cleavable spacer. As the presence of biotin can impair passage of DNA strands through the nanopore, it is preferable for the biotin to be removed from the telomeric DNA fragments prior to nanopore sequencing.

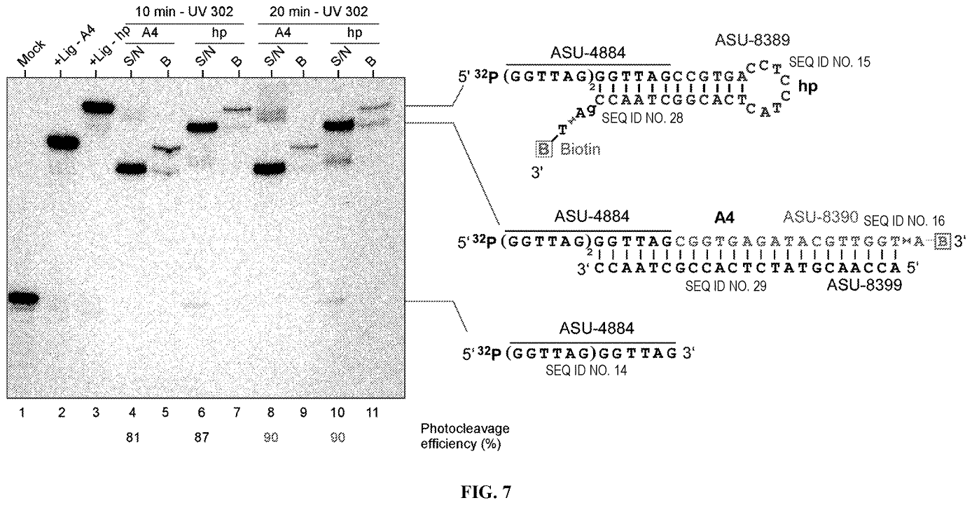

FIG. 6 studies the optimal UV wavelength and UV exposure time to achieve high photocleavage efficiency. Lane 2 shows near complete biotinylated DNA hairpin telomere adapter (ASU-4884) ligation. Streptavidin beads enrichment of ligated DNA products is nearly 100% efficient (lane 3), as any uncaptured ligated DNA cannot be seen in the supernatant (lane 4). The 302 nm UV wavelength shows highly efficient on-bead photocleavage (lanes 6-8) compared to 365 nm (lanes 9-11). Furthermore, 5 min exposure of 302 nm UV exposure is sufficient to achieve high cleavage efficiency (lane 6) and increasing the exposure time does not improve cleavage efficiency (lanes 10-11).

FIG. 7 compares ligation and photocleavage efficiency between the duplex DNA adapter (A4-ASU-8399+ASU-8390) and the biotinylated DNA hairpin telomere adapter (hp-ASU-8389) being ligated to radiolabeled ASU-4884. Schematics of substrate and adapters are shown to the right of the gel showing their gel mobility. Both the hairpin and duplex versions of the telomere adapter ligate with near 100% efficiency (lanes 2 and 3). Following streptavidin bead enrichment of ligated hybrids, the on-bead photocleavage should release the ligated hybrids, and most signal should be detected in the supernatant (S/N) if photocleavage efficiency is high. Photocleavage occurs with more than 80% efficiency as quantified by signal detected from S/N lanes even at 10 min UV 302 nm exposure (lanes 4 and 6) for both biotinylated DNA hairpin telomere adapter and duplex DNA adapter ligated substrates, while the rest remain on beads (lanes 5 and 7). Increasing the UV exposure time to 20 min improves cleavage efficiency by ˜10% (lanes 8 and 10). Thus, photocleavage can be used for both the hairpin and duplex versions of the telomere adapter.

In other implementations, a sufficient amount of telomeric DNA fragments are produced from a single cell for long-read sequencing by amplifying the barcoded and biotinylated telomeric DNA fragment using a high-fidelity and high-processivity DNA polymerase, for example, phi29 DNA polymerase. The forward primer for the amplification reaction has a sequence complementary to the DNA barcode adapter, while the reverse primer has a sequence complementary the telomere adapter (minus the sequence of the single-stranded 3′-overhang) (FIG. 10). In some aspects, the amplification step involves DNA amplification. For example, a 10-cycle of DNA amplification should increase the amount of purified telomeric DNA fragments by 1000 folds, assuming a near 2-fold increase per cycle. The highly processive phi29 DNA polymerase amplification can fully preserve the telomere absolute length which is distinct from the telomere qPCR amplification that use degenerate telomeric primer for targeting all telomeric repeats in the DNA and result in many undesired byproduct and false signals. Accordingly, phi29 DNA polymerase amplification can produce the required minimum amount of 5 fmole of telomeric DNA fragments for nanopore sequencing.

The eluted telomeric DNA fragments or the amplified telomeric DNA fragments are then ligated to a sequencing platform-specific adapter, for example, a nanopore-sequencing adapter or PacBio sequencer adapter, to prepare the eluted telomeric DNA fragments for sequencing. Next, the telomeric DNA fragments are sequenced using a long-read sequencing platform, wherein the read information generated is used to determine the absolute telomere length of each individual chromosome in single cell. In some implementations, the read information from nanopore sequencing is analyzed using a computational program to decode the barcoded reads and to determine the telomere absolute length of each individual chromosome in each single cell sample. In some aspects, the barcoded reads will be sorted and assigned to each of the samples, and each read will be assigned to either p or q arms of each specific chromosome in each DNA sample.

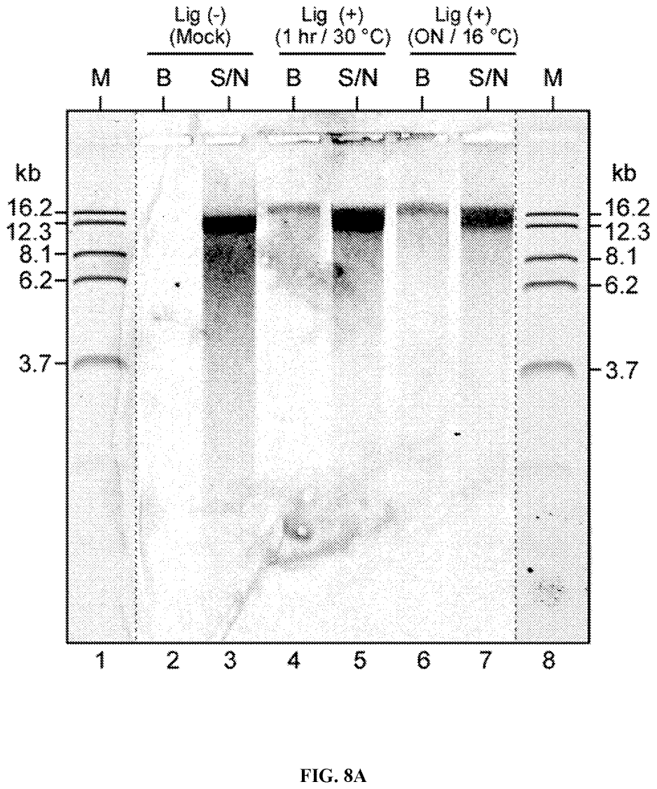

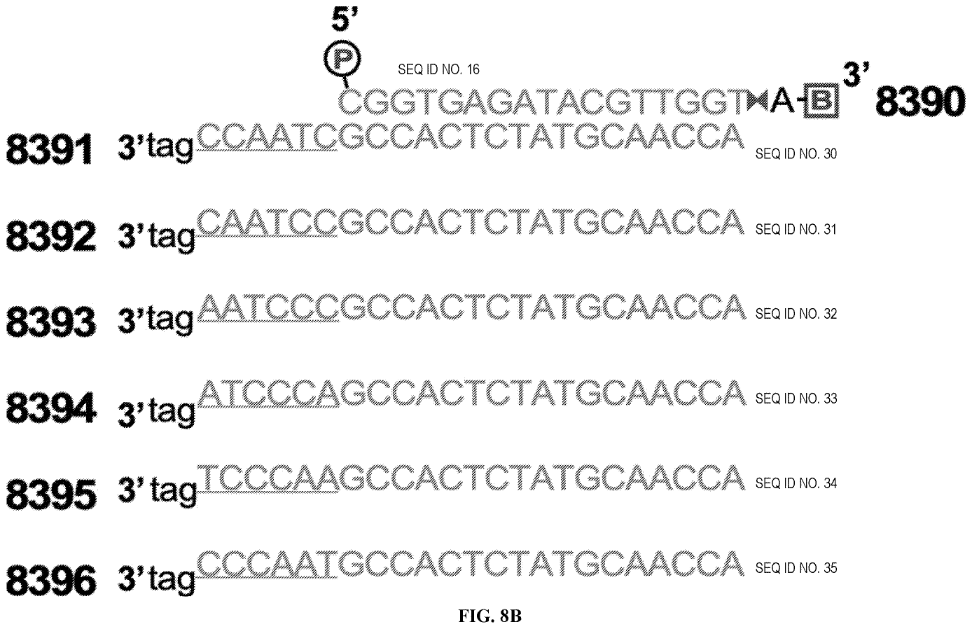

As shown in FIG. 8, human telomeres are enriched following the above described method. Initially after telomere adapter ligation to human genomic DNA, the DNA is restriction digested followed by streptavidin bead enrichment. The enriched telomeres are extracted from the beads and subject to agarose gel-electrophoresis and the telomere signal is detected via probe hybridization seen as diffuse bands on the gel image. In the absence of ligase enzyme, no enrichment can be seen in the beads (B) but is found entirely in the supernatant (lanes 2 and 3). However, 1 hr ligation at 30° C. (lane 4) is sufficient to enrich a significant amount of telomeres comparable to extended ligation time (lane 6).

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Ligation of the Hairpin Form of the Telomere Adapter

First, prepare 100 μl 40 μM stock solutions for each of the six biotinylated DNA hairpin telomere adapters, ASU-7658, ASU-7659, ASU-7660, ASU-7661, ASU-7662, and ASU-7663 (5′-phosphorylated and 3′-biotinylated), in dH₂O. Next, prepare a 500 μl mixture of all 6 adapters at 120 nM oligo mix (20 nM each):

$$\to 970 μl d {H}_{2} O + 5 μl of each 40 μM oligo \times 6 ( 1.2 μM total )$$

$$\to \text{} 50 μl 1.2 μM oligo mix + 450 μl of {d H}_{2} O ( 120 nM total , 20 nM each )$$

The recipe for the ligation reaction (x3) is as follows:

| | | |
|---|---|---|
| 11.5 | μl | 1 μg Genomic DNA #1/#2/#3 (from three sources) |
| 2.0 | μl | 120 nM mixture of each hairpin adapter (final 12 nM total, 2 |
| | | nM each/0.24 pmol total) |
| 4.0 | μl | 5x T4 DNA ligation buffer (Invitrogen) |
| 2.0 | μl | T4 DNA ligase (1 U/μl, Invitrogen) |
| 20.0 | μl | Total |

The ligation reaction is incubated at 30° C. for 1 hour.

### Example 2. Restriction Digestion/Barcoded Adapter Ligation>BamH-I/BcI-I/Bgl-II HF

The recipe for the digestion reaction is as follows:

| | | |
|---|---|---|
| 20.0 | μl | Ligated DNA #1/#2/#3 (from three sources) |
| 9.0 | μl | dH₂O |
| 4.0 | μl | 1 μM barcoded adapter (100 nM final) |
| 4.0 | μl | 10X Cutsmart buffer |
| 1.0 | μl | 10 mM ATP |
| 1.0 | μl | T4 DNA ligase (400 U/μl, NEB) |
| 1.0 | μl | BamH-I HF (20 U/μl)/or other enzymes |
| 40.0 | μl | Total |

The digestion reaction is incubated at 30° C. for 1 hour, and the reaction is terminated with incubation at 65° C. for 10 min. All of the reactions are pooled prior to further processing. For low throughput (1-12 sample), streptavidin purification alone may be sufficient. For high throughput (larger sample numbers, for example, more than 12 samples, between 12-96 samples, or more than 100 samples), an additional purification step may be needed. In such implementations, the sample may be purified using ethanol purification and AMPure XP beads purification.

### 1. Optional AMPure XP Bead Purification

First, the pooled DNA is precipitated using ethanol and then isolated. The AMPure XP beads is resuspended by vortexing prior to being added to the isolated DNA sample. The mixture of beads to sample should be 2:5, so 20 μl AMPure XP beads is added to 50 μl DNA sample. The mixture is rotated at room temperature for 10 min, after which the beads are washed twice with 125 μl Long Fragment Buffer (scale buffer proportionally to initial bead volume).

DNA is eluted from the beads in 20 μl water at 37° C. for 10 min. The eluted DNA (volume of 20 μl) is transferred to a new tube.

### 2. Streptavidin Beads Purification of Biotinylated Telomere DNA

Dynabead MyOne SA-C1 beads (Invitrogen—65001, 10 mg/ml, ˜500 μmol ss-oligo/mg beads) can be used for streptavidin beads purification of biotinylated telomere DNA. Prior to mixing the beads with the DNA sample, the beads are vortexed for 30 sec, and 5 μl C1 beads (50 μg total for 25 μmol biotinylated oligos) are removed for purification of the biotinylated telomere DNA. The beads are first prewashed with three times with 40 μl H₂O and then resuspended in in 80 μl H₂O. For the low throughput method, the pooled hairpin adapter-ligated genomic DNA is added to the resuspended C1 beads (volume of 80 μl). For the high throughput method, the eluted DNA (20 μl) from the purification using AMpure beads is added to the resuspended C1 beads (volume of 80 μl).

The mixture is rotated at room temperature for 30 min. The mixture is then washed once with 200 μl d·H₂O at 50° C. for 1 min. The biotin-labeled telomeric DNA fragments are released from the beads by 300 nm UV irradiation for 10 min, or by incubating the beads in 80 μl of 10 mM EDTA, pH 8.2 with 95% formamide at 65° C. for 5 min. The DNA fragments are isolated using ethanol precipitation and resuspend in 50 μl d·H₂O.

### 3. AMPure XP Bead Purification

The AMPure XP beads are resuspended by vortexing prior to use at a bead: sample ratio of 2:5. Thus, 20 μl AMPure XP beads are added to 50 μl DNA sample. The mixture is rotated at room temperature for 10 min, after which the beads are washed twice with 125 μl Long Fragment Buffer. DNA (#1+ #2+ #3) is eluted from the beads in 30 μl water at 37° C. for 10 min and transferred to a new tube.

### Example 3. Nanopore Sequencing Adapter Ligation

The recipe for ligating the nanopore sequencing adapter is as follows:

| | | |
|---|---|---|
| 30.0 | μl | Pooled barcoded DNA #1/#2/#3 |
| 5.0 | μl | Native Adapter (NA) |
| 10.0 | μl | 5X NEB Quick Ligation Reaction Buffer |
| 5.0 | μl | Quick T4 DNA ligase |
| 50.0 | μl | Total |

The reaction mixture is incubated at room temperature for 20 min. The samples are purified using AMPure XP beads.

20 μl of AMPure XP beads is added to the DNA sample. The mixture is rotated at room temperature for 10 min. The beads are washed once with 125 μl Long Fragment Buffer. DNA is eluted from the beads in 15 μl Elution Buffer at 37° C. for 10 min and transferred to a new tube. OF the 15 μl volume, 1 μl is used for Tapestation analysis (or Qubit fluorometer). The library is adjusted to 12 μl at 10-20 fmol (roughly 0.2 μg DNA of average length of 25 kb) for nanopore sequencing. For example, the samples are primed and loaded on SpotON Flow cell using standard protocol.

### Example 4. Measuring Telomere Absolute Length in a Single Cell

The method can be modified to measure individual telomere absolute length in single cells using a high-throughput format combining DNA samples prepared from a large number of single cells. A population of suspension cells will be sorted and dispensed in each well of a 96- or 384-well plates using a flow cytometry cell sorter. Each cell will be lysed to release genomic DNA which will be processed to be biotinylated, barcoded, and purified. Thus, genomic DNA from a single cell will receive the same barcode, and different barcodes indicate telomere measurements for a single cell. All purified barcoded telomeric DNA fragments will be pooled and amplified by polymerase chain reaction using a highly processive phi29 DNA polymerase enzyme and two primers that target both end of the telomeric DNA fragment at the barcode adapter and hairpin adapter. Since the phi29 DNA polymerase is not thermophilic, the polymerase chain reaction will be performed manually by adding 1 μl of phi29 DNA polymerase after each cycle of denaturation and primer annealing. A microfluidic device can be designed for adding the phi29 DNA polymerase per cycle. It is estimated that 10 cycles of the Phi29 amplification will produce over 100-fold more telomeric DNA fragments for nanopore sequencing. An aliquot of 5 fmole amplified telomeric DNA fragments will be loaded to a nanopore flow cell for long-read sequencing analysis.

### Example 5. Exemplary Sequencing Data Providing Absolute Length of Individual Telomeres from Three Cell Lines

Telomere lengths from three different human cell lines (BJ-hTERT, Hela, and Jurkat) were measured in a pilot experiment using six telomere adapters in the form of Design 1 in FIG. 4, but without a photocleavable spacer attaching the biotin. Genomic DNA samples were extracted from the three human cell lines and ligated to a set of six biotinylated DNA hairpin adapters and specific DNA barcode adapters, BA1, BA2 and BA3, respectively. All three biotinylated and barcoded telomeric DNA fragments were pooled and ligated to the nanopore sequencing adapter and analyzed by nanopore long-read sequencing. After computational analysis, a small number of reads containing telomeric sequence were obtained. The individual telomere absolute length (iTAL) was determined for each read (see Table 2). The average iTALs from the three cell lines are consistent with the previously reported telomere length measured by TRF (telomeric restriction fragment) assay, which validates the disclosed method as proof of principle. Table 3 shows the telomere calculations from exemplary individual reads. A distinct barcode was provided for each cell line. The variation in the selected barcode indicates the barcode sequence does not affect the measurement of telomere lengths.

TABLE 2
Measurements individual telomere absolute
length (iTAL) in three human cell lines.

| | | | Telomere | |
|---|---|---|---|---|
| Cell | | Average | length | |
| line | iTAL (bp) | iTAL | reported | |
| BJ- | 7264, 7155, 6756, 6543, 6065, 5841, | 6481 bp | ~7.3 | kb |
| hTERT | 5745, 2690, 1805, 735, 548 | | | |
| Hela | 6066, 5670, 5617, 2954, 2762, 2677, | 4291 bp | ~5 | kb |
| | 1919, 1131, 377 | | | |
| Jurkat | 8755, 6489, 5760, 4372, 3765, 2622, | 4414 bp | ~5.1 | kb |
| | 1995, 1555, 1358, 1147, 822 | | | |

TABLE 3
Telomere length measurements based on Read ID in cells
from three human cell lines.

| | | Barcode | | | Telomere |
|---|---|---|---|---|---|
| Read ID | Barcode | seq | Cell line | Enzyme | length (bp) |
| 853ccd8a-f75a-4f4f-b1bc- | BA-1 | CGTCTA | BJ- | Bcl-I | 7264 |
| b9a9a645064d | | | hTERT | | |
| ab1ec90b-1013-40c6-9c25- | BA-1 | CGTCTA | BJ- | Bcl-I | 7155 |
| 73d60fa0ee06 | | | hTERT | | |
| 5322eb42-87cb-4b72-aa8d- | BA-1 | CGTCTA | BJ- | Bcl-I | 6756 |
| 9e700ad16221 | | | hTERT | | |
| b0583194-0e52-47dd-b481- | BA-1 | CGTCTA | BJ- | Bcl-I | 6543 |
| 213221028b98 | | | hTERT | | |
| c1fe964d-5ed0-4636-a718- | BA-1 | CGTCTA | BJ- | Bcl-I | 6065 |
| 038af1a94fbd | | | hTERT | | |
| b58ae1df-162d-4cf6-8bdd- | BA-1 | CGTCTA | BJ- | Bcl-I | 5841 |
| 395ff5b137b1 | | | hTERT | | |
| 30a49778-a67e-44d3-ad9e- | BA-1 | CGTCTA | BJ- | BamHI | 5745 |
| f33b486043a6 | | | hTERT | | |
| 6c8cb9ca-1576-45e4-b6f8- | BA-1 | CGTCTA | BJ- | Bcl-I | 2690 |
| 4670de197da6 | | | hTERT | | |
| 72ab9a3b-77d4-4c1d-972c- | BA-1 | CGTCTA | BJ- | Bcl-I | 1805 |
| 0498d0c55640 | | | hTERT | | |
| 63c2c3d0-9c8d-4768-81f2- | BA-1 | CGTCTA | BJ- | Bcl-I | 735 |
| 9ba4bd231bc6 | | | hTERT | | |
| dc66442e-acbd-4ed7-ba38- | BA-1 | CGTCTA | BJ- | Bcl-I | 548 |
| 8167b57622a5 | | | hTERT | | |
| 21fff8a0-07c5-4350-bc4d- | BA-2 | CGCTTA | HeLa | Bcl-I | 6066 |
| 84b6603af2f4 | | | | | |
| b82f10eb-0c1c-4abc-aab3- | BA-2 | CGCTTA | HeLa | Bcl-I | 5670 |
| 39dc18a52056 | | | | | |
| 6b8b7a87-0c6b-404d-a04f- | BA-2 | CGCTTA | HeLa | Bcl-I | 5617 |
| 95d11eb36b96 | | | | | |
| a147d0bd-fd0e-4502-bc45- | BA-2 | CGCTTA | HeLa | Bcl-I | 2954 |
| 69b396117d72 | | | | | |
| 67621ac1-6ce9-4a5c-a344- | BA-2 | CGCTTA | HeLa | Bcl-I | 2762 |
| 8b78165258df | | | | | |
| fd24c11a-4559-4c3c-a146- | BA-2 | CGCTTA | HeLa | Bcl-I | 2677 |
| aa79e544fb8b | | | | | |
| 38ad734f-63c0-4ea9-a705- | BA-2 | CGCTTTA | HeLa | Bcl-I | 1919 |
| 523866b99cab | | | | | |
| e324f95e-eafa-46bb-a88f- | BA-2 | CGCTTA | HeLa | Bcl-I | 1139 |
| b0c7c683fd26 | | | | | |
| 3c36a0a8-0c41-40f4-bdf1- | BA-2 | CGCTTA | HeLa | Bcl-I | 377 |
| 05399217844c | | | | | |
| d77e6722-3268-4568-bed4- | BA-3 | CGTCAT | Jurkat | Bcl-I | 8755 |
| 296c352c5f5b | | | | | |
| 52a87ela-7b6f-4e50-b2af- | BA-3 | CGTCAT | Jurkat | Bcl-I | 6489 |
| 07098e3cbadf | | | | | |
| 7d9164b9-54a5-47b1-98bb- | BA-3 | CGTCAT | Jurkat | Bcl-I | 5760 |
| 8d5109bcd1b4 | | | | | |
| 32154abe-32b6-45b5-a771- | BA-3 | CGTCAT | Jurkat | Bcl-I | 4372 |
| 5358ff60eb33 | | | | | |
| 8554a330-664b-455a-9055- | BA-3 | CGTCAT | Jurkat | Bcl-I | 3765 |
| 36e05ac18b06 | | | | | |
| 3c9120ad-33cd-4e18-ad59- | BA-3 | CGTCAT | Jurkat | Bcl-I | 2622 |
| 85604080a386 | | | | | |
| d130ac10-f095-4535-ae6c- | BA-3 | CGTCAT | Jurkat | Bcl-I | 1995 |
| 474942ad50bc | | | | | |
| 4c045cad-d959-414f-8ed6- | BA-3 | CGTCAT | Jurkat | Bcl-I | 1555 |
| 5c199cbfba5d | | | | | |
| 73c5cf2d-aaf7-49c5-9b75- | BA-3 | CGTCAT | Jurkat | Bcl-I | 1358 |
| 797ecc88e29e | | | | | |
| 5048f441-ac51-4ef6-99cb- | BA-3 | CGTCAT | Jurkat | Bcl-I | 1147 |
| f69231393321 | | | | | |
| 14c0d4b1-6762-4cac-875b- | BA-3 | CGTCAT | Jurkat | Bcl-I | 822 |
| 43e4e8f82869 | | | | | |

Bolded letters in the Barcode seq column indicate substitutions.
Underlined letters in the Barcode seq column indicate deletions. Bolded and underlined letters in the Barcode seq column indicate insertions.

However, the number of reads containing telomeric sequence was unexpected low, which was likely due to the bulky biotin at the 3′-end of the purified telomeric DNA fragments. The inclusion of a cleavable spacer in the biotin DNA adapter would allow the removal of the biotin after photo-elution of the telomeric DNA fragments from the streptavidin beads. The bulky biotin is problematic only for the nanopore sequencing platform but not for the PacBio platform.

What is claimed:

1. A method of determining absolute telomere length comprising:
providing a genomic DNA sample, wherein the genomic DNA sample comprises telomeric DNA fragments;
ligating a set of six telomere biotin-DNA adapters with the telomeric DNA fragments to produce a ligation product comprising biotinylated telomeric DNA fragments, wherein:
the telomere biotin-DNA adapter comprises biotin attached via a cleavable spacer;
the telomere biotin-DNA adapter comprises a single-stranded 3′-overhang, which comprises a complementary to the 3′ end of the telomeric DNA fragments and a 5′ phosphate for ligation with the telomeric DNA fragments; and
the set of six telomere biotin-DNA adapters comprise:
a first telomere adapter with its single-stranded 3′-overhang comprising CCAATC (3′ to 5′);
a second telomere adapter with its single-stranded 3′-overhang comprising CAATCC (3′ to 5′);
a third telomere adapter with its single-stranded 3′-overhang comprising AATCCC (3′ to 5′);
a fourth telomere adapter with its single-stranded 3′-overhang comprising ATCCCA (3′ to 5′);
a fifth telomere adapter its single-stranded 3′-overhang comprising TCCCAA (3′ to 5′); and
a sixth telomere adapter with its single-stranded 3′-overhang comprising CCCAAT (3′ to 5′);
digesting the ligation product with a restriction enzyme to release the biotinylated telomeric DNA fragments from the non-telomeric genomic DNA;
ligating a DNA barcode adapter to the biotinylated telomeric DNA fragments at the restriction enzyme digestion site to produce barcoded and biotinylated telomeric DNA fragments;
cleaving the biotin from the barcoded and biotinylated telomeric DNA fragments to produce barcoded telomeric DNA fragments; and
sequencing the barcoded telomeric DNA fragments.

2. The method of claim 2, wherein the telomeric biotin-DNA adapters are biotinylated DNA hairpins, wherein the biotinylated DNA hairpin has a stem-loop structure comprising a double-stranded stem and a single-stranded loop and the single-stranded 3′-overhang, wherein a biotin is attached at the 3′ end.

3. The method of claim 2, wherein the single-stranded 3′-overhang is at least 6 nucleotides in length.

4. The method of claim 2, wherein the telomeric biotin-DNA adapters are duplex DNAs, wherein the duplex DNA comprises a first oligonucleotide and a second oligonucleotide, wherein:
the duplex DNA comprises an annealed region of 12-18 bp, wherein the first oligonucleotides and the second oligonucleotide are perfectly complementary;
the Tₘ of the annealed region is >30° C.;
the second oligonucleotide is longer than the first oligonucleotide thereby providing the single-stranded 3′-overhang; and
the first oligonucleotide comprises a 5′ phosphate.

5. The method of claim 4, wherein the biotin is attached to the 3′ end of the first oligonucleotide.

6. The method of claim 4, wherein the biotin is attached to the 5′ end of the second oligonucleotide.

7. A method of determining the absolute telomere length in a single cell comprising:
isolating genomic DNA from a single cell to provide a genomic DNA sample comprising telomeric DNA fragments;
ligating a set of six telomere adapters with the telomeric DNA fragments to produce a ligation product comprising biotinylated telomeric DNA fragments, wherein:
the telomere adapter comprises a single-stranded 3′-overhang, which comprises a complementary to the 3′ end of the telomeric DNA fragments and a 5′ phosphate for ligation with the telomeric DNA fragments; and
the set of six telomere adapters comprise:
a first telomere adapter with its single-stranded 3′-overhang comprising CCAATC (3′ to 5′);
a second telomere adapter with its single-stranded 3′-overhang comprising CAATCC (3′ to 5′);
a third telomere adapter with its single-stranded 3′-overhang comprising AATCCC (3′ to 5′);
a fourth telomere adapter with its single-stranded 3′-overhang comprising ATCCCA (3′ to 5′);
a fifth telomere adapter its single-stranded 3′-overhang comprising TCCCAA (3′ to 5′); and
a sixth telomere adapter with its single-stranded 3′-overhang comprising CCCAAT (3′ to 5′);
digesting the ligation product with a restriction enzyme to release the biotinylated telomeric DNA fragments from the non-telomeric genomic DNA;
ligating a DNA barcode adapter to the biotinylated telomeric DNA fragments at the restriction enzyme digestion site to produce barcoded and biotinylated telomeric DNA fragments;
amplifying the barcoded and biotinylated telomeric DNA fragments with phi29 DNA polymerase with a forward primer comprising a sequence complementary to the DNA barcode adapter, and a reverse primer comprising a sequence complementary a portion of the telomere adapter lacking single-stranded 3′-overhang; and
sequencing the amplified telomeric DNA fragments.

8. The method of claim 7, wherein the steps of ligating the biotinylated DNA hairpins, digesting the ligation product, and ligating the DNA barcode are performed in a single reaction vessel.

9. The method of claim 7, wherein the telomeric adapters are DNA hairpins, wherein the DNA hairpin has a stem-loop structure comprising a double-stranded stem and a single-stranded loop and the single-stranded 3′-overhang.

10. The method of claim 7, wherein the single-stranded 3′-overhang is at least 6 nucleotides in length.

11. The method of claim 7, wherein the telomeric adapters are duplex DNAs, wherein the duplex DNA comprises a first oligonucleotide and a second oligonucleotide, wherein:
the duplex DNA comprises an annealed region of 12-18 bp, wherein the first oligonucleotides and the second oligonucleotide are perfectly complementary;
the Tₘ of the annealed region is >30° C.;
the second oligonucleotide is longer than the first oligonucleotide thereby providing the single-stranded 3′-overhang; and
the first oligonucleotide comprises a 5′ phosphate.

12. The method of claim 11, wherein the biotin is attached to the 3′ end of the first oligonucleotide.

13. The method of claim 11, wherein the biotin is attached to the 5′ end of the second oligonucleotide.

14. The method of any one of claims 1-13, wherein the concentrations of the individual telomere biotin-DNA adapter or the individual telomere adapters are the same.

15. The method of claim 14, wherein the total concentration of six telomere biotin-DNA adapters or the total concentration of the six telomere adapters is 3-4 nM.

16. The method of claim 14, wherein the total concentration of the set of six telomere biotin-DNA adapters or the total concentration of the six telomere adapters is about 3 nM in total.

17. The method of any one of claims 1-13, wherein the restriction enzyme is selected from BamH-I, BcI-I, Bgl-II HF, and NotI.

18. The method of claim 17, wherein:
the first telomere adapter comprises the sequence set forth in SEQ ID NO. 8 or has the sequence set forth in SEQ ID NO. 21X;
the second telomere adapter comprises the sequence set forth in SEQ ID NO. 9 or has the sequence set forth in SEQ ID NO. 22X;
the third telomere adapter comprises the sequence set forth in SEQ ID NO. 10 or has the sequence set forth in SEQ ID NO. 23X;
the fourth telomere adapter comprises the sequence set forth in SEQ ID NO. 11 or has the sequence set forth in SEQ ID NO. 24X;
the fifth telomere adapter comprises the sequence set forth in SEQ ID NO. 12 or has the sequence set forth in SEQ ID NO. 25X; and
the sixth telomere adapter comprises the sequence set forth in SEQ ID NO. 13 or has the sequence set forth in SEQ ID NO. 26X.

19. The method of claim 17, wherein the barcoded telomeric DNA fragments or the amplified telomeric DNA fragments are sequenced using nanopore sequencing.

20. The method of claim 17, further comprising determining the absolute length of telomeres in the genomic DNA sample from the nanopore sequencing results.

21. The method of any one of claims 1-13, wherein the DNA barcode adapter comprise a compatible sticky end to the end created from restriction enzyme digestion.

22. The method of claim 21, wherein the restriction enzyme is selected from BamH-I, BcI-I, Bgl-II HF, and NotI.

23. The method of claim 22, wherein the barcoded telomeric DNA fragments or the amplified telomeric DNA fragments are sequenced using nanopore sequencing.

24. The method of claim 22, further comprising determining the absolute length of telomeres in the genomic DNA sample from the nanopore sequencing results.
